(19) United States
(12) Patent Application Publication
(10) Pub. No.: US 2026/0311885 A1
(43) Pub. Date: Oct. 8, 2026

(54) PREDICTING MOTOR FUNCTION SCORES IN POMPE DISEASE TREATMENT

(71) Applicant: Genzyme Corporation, Cambridge, MA (US)

(72) Inventors: Julie L. Batista, Cambridge, MA (US); Kelly George, Cambridge, MA (US); Kristina An Haack, Cambridge, MA (US); Rana Jreich, Cambridge, MA (US); Chanchala Kaddi, Cambridge, MA (US); Zhaoling Meng, Cambridge, MA (US); Catherine Ortemann-Renon, Cambridge, MA (US); Fatiha Rachedi, Cambridge, MA (US); Mengdi Tao, Cambridge, MA (US); Atef Zaher, Cambridge, MA (US); Susana Zaph, Cambridge, MA (US)

(21) Appl. No.: 19/481,938

(22) PCT Filed: May 10, 2024
(86) PCT No.: PCT/US2024/028764

§ 371 Date: Nov. 6, 2025

Related U.S. Application Data
(60) Provisional application No. 63/502,037, filed on May 12, 2023.
(60) Provisional application No. 63/624,935, filed on Jan. 25, 2024.

(51) Int. Cl. G16H 50/30; G16C 20/30

(52) U.S. Cl. CPC G16H 50/30; G16C 20/30

(57) ABSTRACT

A prediction method for predicting a change in the motor function score from a baseline score. The system obtains input data including a temporal trajectory of an indicator metabolite compound for the subject. The determines a predicted temporal trajectory of the motor function score using a prediction model and the input data.

### CROSS-REFERENCE TO RELATED APPLICATION

This application is the national stage entry of International Patent Application No. PCT/US2024/028764, filed on May 10, 2024, which claims priority to U.S. Provisional Patent Application No. 63/502,037, filed on May 12, 2023, and U.S. Provisional Patent Application No. 63/624,935, filed on Jan. 25, 2024, the disclosures of both of which are hereby incorporated by reference in its entirety.

### TECHNICAL FIELD

This specification generally relates to predicting motor functions of a subject based on a biomarker.

### BACKGROUND

This specification generally relates to predicting a motor function score for Pompe disease treatment.

Motor scores, such as the Gross Motor Function Measure (GMFM) and the Quick Motor Function Test (QMFT), have been used as efficacy endpoints to evaluate motor function in the treatment of diseases that affect motor function. For instance, GMFM and QMFT scores are employed in assessing the effectiveness of treatments for Pompe disease.

Pompe disease is a rare recessive disorder characterized by progressive, debilitating, and often fatal neuromuscular symptoms that affect multiple systems. It is caused by pathogenic variants of the GAA gene, resulting in a deficiency of acid alpha-glucosidase (GAA) enzyme activity and the progressive accumulation of glycogen in lysosomes.

Pompe disease is categorized into two forms: Infantile-Onset Pompe Disease (IOPD) and Late-Onset Pompe Disease (LOPD). The disease presents as a spectrum in terms of onset and progression. In IOPD, symptoms typically manifest before or at 12 months of age and include cardiomyopathy. In contrast, LOPD patients either have symptom onset after 12 months of age or before 12 months without cardiomyopathy.

Myozyme (alglucosidase alfa) is a globally approved enzyme replacement therapy (ERT) for the entire spectrum of Pompe disease, while Nexviazyme (avalglucosidase alfa) is approved for LOPD and for IOPD in the EU, but only for LOPD in the US.

The change from baseline in the GMFM-88 percent and/or the QMFT raw total scores have been utilized as efficacy endpoints for assessing motor function in clinical trials for both IOPD and LOPD populations with Pompe disease. GMFM-88 percent and QMFT raw total scores changes from baseline over time are influenced by the patient's initial functional status.

The urinary biomarker hexose tetrasaccharide (urine Hex4 (mmol/mol)) can serve as an indirect measure of the extent of skeletal muscle glycogen clearance in Pompe disease.

### SUMMARY

This disclosure describes methods, computer systems, and apparatus, including computer programs encoded on computer storage media, for predicting a motor function score that measures one or more motor functions of a subject during the treatment of Pompe disease.

In one aspect, this disclosure provides a prediction method for predicting a change in the motor function score from a baseline score. The motor function score measures one or more motor functions of a subject. The method can be implemented by a system including one or more computers. The system obtains input data including a temporal trajectory of an indicator metabolite compound for the subject. The temporal trajectory of the indicator metabolite compound including, for each of one or more time points, a respective value for the indicator metabolite compound for the subject at the respective time point. The system obtains a plurality of first model parameters of a prediction model, and determines a predicted temporal trajectory of the motor function score using the input data. The determining includes: for each of the one or more time points after a baseline time point, determining a respective predicted value for a change in the motor function score from a baseline value at the respective time point using the prediction model based at least on (i) the input data and (ii) the first model parameters. The prediction model includes a non-linear functional term of a change of the respective value for the indicator metabolite compound at the respective time point from a baseline value. The system then outputs data characterizing the predicted temporal trajectory of the motor function score.

In some implementations of the prediction method, the prediction model is a non-linear random coefficient model that includes one or more random parameters. In some cases, a rate of change parameter in the non-linear functional term is one of the random parameters. In some cases, to determine the respective predicted value, for each of the one or more random parameters, the system selects a respective value for the respective random parameter from a respective pre-defined distribution, wherein the predicted value is determined based on the respective values selected for the one or more random parameters.

In some implementations of the prediction method, the non-linear functional term is a decaying exponential functional term. In some cases, the prediction model is a non-linear random coefficient model, and a negative change rate parameter in the exponential functional term is a random parameter. In some cases, an asymptote parameter of the exponential functional term is a fixed parameter.

In some implementations of the prediction method, the motor function score is a Gross Motor Function Measure (GMFM) score.

In some implementations of the prediction method, the motor function score is a Quality of Upper Extremity Skills Test (QMFFT) score.

In some implementations of the prediction method, the indicator metabolite compound is urinary hexose tetrasaccharide (uHex4), and the value for the indicator metabolite compound is a uHEX4 concentration value. In some cases, one or more of the respective uHex4 concentration values are obtained through measurement of the uHex4 concentration at the respective time points for the subject.

In some implementations of the prediction method, the input data further includes (i) one or more demographic attributes of the subject, (ii) one or more clinical diagnoses for the subject, or (iii) a treatment history of the subject.

In some implementations of the prediction method, the respective predicted value is determined further based on (i) the baseline score for the subject and (ii) a median baseline score obtained for a population of subjects.

In some implementations of the prediction method, the predicted value is determined further based on (i) an age of diagnosis of the subject and (ii) a median age of diagnosis obtained for a population of subjects.

In some implementations of the prediction method, the respective predicted value is determined further based on a median baseline value for the indicator metabolite compound obtained for a population of subjects.

In some implementations of the prediction method, the subject is a subject that has been diagnosed with a particular disease that affects motor functions. In some cases, the particular disease is Infantile-onset Pompe disease (IOPD). In some cases, the particular disease is Late-onset Pompe disease (LOPD).

In some implementations of the prediction method, the subject is under an enzyme replacement therapy for Pompe disease. In some cases, the therapy is an alpha-glucosidase replacement therapy. In some cases, the therapy is an avalglucosidase alfa replacement therapy.

In some implementations of the prediction method, obtaining the temporal trajectory of the indicator metabolite compound for the subject includes: obtaining a quantitative systems pharmacology (QSP) model of Pompe disease metabolism; and predicting the temporal trajectory of the value of the indicator metabolite compound as a biomarker of disease activity using the QSP model at each of the one or more time points.

In some implementations of the prediction method, the subject is a simulated subject that represents a real subject having been diagnosed with a form of Pompe disease. In some cases, the form of Pompe disease is an Infantile-onset Pompe disease (IOPD). In some cases, the form of Pompe disease is a Late-onset Pompe disease (LOPD).

In some cases, the QSP model is used to simulate an enzyme replacement therapy for the simulated subject. In some cases, the enzyme replacement therapy is an alpha-glucosidase replacement therapy. In some cases, the enzyme replacement therapy is an avalglucosidase alfa replacement therapy.

In some implementations of the prediction method, the system further determines a treatment efficacy of the enzyme replacement therapy based at least on the predicted temporal trajectory of the motor function score for the simulated subject. In some cases, the system further determines whether a particular real subject should receive the enzyme replacement therapy based at least on the respective predicted temporal trajectories of the motor function scores for the simulated subject. In some cases, in response to determining that the particular real subject should receive the enzyme replacement therapy, the enzyme replacement therapy is physically administered to the particular real subject.

In some implementations of the prediction method, predicting the temporal trajectory of the value of the indicator metabolite compound using the QSP model includes: obtaining a set of subject-specific model parameters for the subject for the QSP model; and computing the respective temporal trajectory of the value of the indicator metabolite compound for the subject using the QSP model based on the set of subject-specific model parameters.

In some implementations of the prediction method, the set of subject-specific model parameters for the subject is determined by: adjusting one or more of a set of population-based model parameters based on one or more of: (i) one or more demographic attributes of the subject, (ii) one or more laboratory test results from the subject, (iii) one or more one clinical diagnosis for the subject, or (iv) a treatment history of the subject. In some cases, the set of population-based model parameters are determined for the QSP model based on a first benchmarking dataset for a population of subjects. In some cases, the population of subjects include subjects having been diagnosed with the IOPD. In some cases, the population of subjects include subjects having been diagnosed with the LOPD.

In another aspect, this disclosure provides a parameter estimation method for determining model parameters of a prediction model for predicting a change in a motor function score from a baseline score. The parameter estimation method can be implemented by a system including one or more computers. The system obtains a second benchmarking dataset including, for each of a plurality of subjects, (i) respective input data specifying, for each of a plurality of time points, a respective value for a change in an indicator metabolite compound for the subject at the respective time point, and (ii) respective output data specifying, for each of the plurality of time points, a respective output value for the change in the motor function score for the subject at the respective time point. The system determines the model parameters for the prediction model based on the second benchmarking dataset, wherein the prediction model includes a non-linear functional term of a change in the value of the indicator metabolite compound for a subject, and outputs the model parameters.

In some implementations of the parameter estimation method, the motor function score is a Gross Motor Function Measure (GMFM) score.

In some implementations of the parameter estimation method, the motor function score is a Quality of Upper Extremity Skills Test (QMFT) score.

In some implementations of the parameter estimation method, the indicator metabolite compound is urinary hexose tetrasaccharide (uHex4), and the value for the indicator metabolite compound is a uHEX4 concentration value.

In some implementations of the parameter estimation method, the prediction model is a non-linear random coefficient model that includes one or more random parameters. In some cases, a rate of change parameter in the non-linear functional term is one of the random parameters.

In some implementations of the parameter estimation method, the non-linear functional term is a decaying exponential functional term. In some cases, the prediction model is a non-linear random coefficient model, and a negative change rate parameter in the exponential functional term is a random parameter. In some cases, an asymptote parameter of the exponential functional term is a fixed parameter.

In some implementations of the parameter estimation method, the prediction model further includes a term based on (i) a baseline score for the subject and (ii) a median baseline score obtained for a population of subjects.

In some implementations of the parameter estimation method, the prediction model further includes a term based on (i) an age of diagnosis of the subject and (ii) a median age of diagnosis obtained for a population of subjects.

In some implementations of the parameter estimation method, the prediction model further includes a term based on a median baseline value for the indicator metabolite compound obtained for a population of subjects.

This disclosure also provides a system including one or more computers and one or more storage devices storing instructions that, when executed by the one or more computers, cause the one or more computers to perform the methods described above.

This disclosure also provides one or more computer storage media storing instructions that when executed by one or more computers, cause the one or more computers to perform the methods described above.

The subject matter described in this disclosure can be implemented in particular embodiments so as to realize one or more advantages.

The described techniques provide techniques for predicting the motor function scores of a subject over a period of time. For example, the described technique can allow the use of the urinary hexose tetrasaccharide (uHex4), which is an indirect measure of the degree of skeletal muscle glycogen clearance in Pompe disease, as a biomarker to estimate the improvement in the motor function score of a patient under a particular treatment.

The provided prediction techniques can offer a cost-effective and time-efficient alternative to repeated direct measurement of motor functions, which may require extensive clinical assessments and/or specialized equipment. The measurement of the uHex4 marker can be performed in a laboratory setting with relatively faster turnaround times and lower costs compared to comprehensive motor function assessments. As a result, more frequent assessment can be performed in the treatment duration to help researchers and clinicians make informed predictions about the improvement of motor functions of the patient, and thus help to guide treatment decisions and monitor disease progression more effectively.

In particular, some implementations of the described techniques combine a quantitative systems pharmacology (QSP) model and a prediction model to predict Pompe disease treatment outcomes for different treatment approaches. The QSP model can integrate diverse data including data from clinical studies, real-world datasets, biological pathways, as well as personalized data of individual patients with mathematical representations of the Pompe pathophysiology to simulate the enzyme deficiency that characterizes Pompe disease and the resulting biomarker profile. The prediction model links biomarkers (e.g., uHex4 concentrations) with clinical outcomes (e.g., motor function indicators). The combination of the QSP model and the prediction model provides a toolbox for personalized care and more effective drug development for Pompe disease.

For example, the provided techniques enable directly comparing outcomes of different treatment options, e.g., avalglucosidase alfa and alglucosidase alfa, under controlled conditions for Pompe disease. Traditionally, comparing the effectiveness of different treatment options often involves clinical trials with their inherent limitations, such as patient heterogeneity and sample size constraints. The described techniques use the QSP model to create simulated patients who represent real individuals and allow for head-to-head comparisons between different treatment options. This eliminates the need for additional physical trials, reducing risks for patients and potentially accelerating research efforts.

In another example, by creating “virtual twins” of real patients, the described techniques can be used to predict how a specific patient might respond to each treatment option based on their unique demographics. This opens doors for personalized treatment plans, tailoring therapy to maximize individual benefit and minimize potential side effects.

### BRIEF DESCRIPTION OF THE DRAWINGS

FIG. 1 shows a workflow of an example prediction system for predicting the motor function of a subject.

FIG. 2 shows an example QSP model for describing cellular metabolism in Pompe disease.

FIG. 3 is a flow diagram illustrating an example process for predicting the motor function of a subject.

FIG. 4 illustrates the performance of the motor function score prediction model.

FIG. 5 illustrates the performance of a QSP model.

FIG. 6 is a block diagram of an example computer system.

Like reference numbers and designations in the various drawings indicate like elements.

### DETAILED DESCRIPTION

Evaluating the motor function of a subject is crucial in several areas of medicine and research. For example, in diseases that cause muscle weakness or movement difficulties, like Pompe disease, evaluating the temporal trajectories of motor function change helps doctors assess disease severity, progression, and/or treatment efficacy. Furthermore, evaluating motor function is essential during the development of new therapies or interventions. Clinical trials often use changes in motor function scores as a primary outcome to measure treatment effectiveness. Accurate evaluation and prediction help researchers design better trials, choose appropriate sample sizes, and determine the optimal therapeutic interventions and optimal length of time to monitor patients for meaningful results.

While directly measuring motor function through clinical assessments is crucial, it can be inefficient and impractical for several reasons. Firstly, these assessments can be time-consuming and require specialized equipment and trained personnel, limiting the frequency with which they can be performed or the number of patients that can be monitored in clinical trials.

Secondly, direct measurements cannot be performed for hypothetical treatments, while researchers or clinicians may need to estimate how patients might respond to a particular therapy before applying the therapy or compare the outcome from multiple potential treatment options.

This specification describes techniques for predicting motor function based on biomarkers such as urinary hexose tetrasaccharide (uHex4) in Pompe disease. The described prediction techniques provide a way to track disease progression and potentially estimate a patient's response to treatment without frequently using time-consuming and expensive clinical assessments of the patient's motor function.

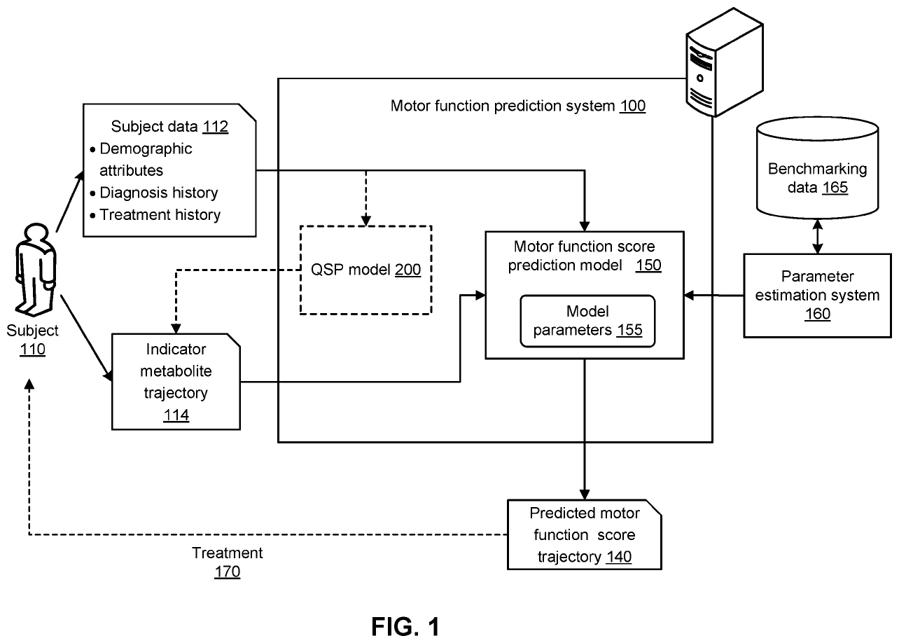

FIG. 1 shows a workflow of an example prediction system 100 for predicting the motor function of a subject 110. The prediction system 100 is an example of a system implemented as computer programs on one or more computers in one or more locations in which the systems, components, and techniques described below are implemented.

The prediction system 100 includes a prediction model 150. The prediction system 100 uses the prediction model 150 to process data including (i) subject data 112 and (ii) a temporal trajectory of an indicator metabolite 114 to predict a temporal trajectory of a motor function score 140 of the subject. In particular, at each time point after a baseline time point, the prediction model 150 predicts a change in the motor function score from a baseline score based on a measurement of the indicator metabolite at that time point.

The time points used to track the temporal trajectory can be customized to specific research or clinical needs. The trajectory can cover days, weeks, months, or years, with time points taken at intervals of days, weeks, or months accordingly.

The motor function score can be any appropriate metric measuring one or more motor functions of the subject 110. In some cases, the motor function score is a Gross Motor Function Measure (GMFM) score. For example, it can be the GMFM-88 percent total score or a GMFM-66 percent total score. In some other cases, the motor function score can be a Quality of Upper Extremity Skills Test (QMFT) score. Some other examples of the motor function score include the Timed Up and Go test (TUG) score and the Nine-Hole Peg Test (NHPT) score.

The prediction system 100 outputs the predicted motor score trajectory 140. In some cases, the predicted motor score trajectory 140 can be used to monitor the progression of a disease that affects motor function. In some cases, the predicted motor score trajectory 140 can be used to determine the effectiveness of a treatment 170 administered to the subject 110, and adjust the treatment strategy. For example, the prediction system 100 can be used to generate the predicted motor score trajectory 140 for the subject 110 before and after the treatment started. After the subject receives the treatment, an increase in the predicted motor score would suggest the effectiveness of the treatment.

In some cases, the predicted motor score trajectory 140 can be used to select a particular treatment 170 for the subject 110. In a particular example, for a patient having been diagnosed with Pompe disease, the prediction system 100 or another system can determine the treatment efficacy of an enzyme replacement therapy (e.g., an alpha-glucosidase replacement therapy or an avalglucosidase alfa replacement therapy) based on least on the predicted motor score trajectory 140. In another example, the prediction system 100 or another system can determine whether the subject 110 should receive the enzyme replacement therapy based at least on the predicted motor score trajectory 140. In response to determining that the subject 110 should receive the enzyme replacement therapy 170, the treatment will be physically administered to the subject 110.

The subject-specific data 112 can include data characterizing the subject 110, and include data such as demographic attributes (e.g., age and gender), diagnosis history (e.g., age of diagnosis), treatment history (e.g., duration of previous treatment), and clinical data (e.g., the body mass index, the creatine kinase levels). The subject-specific data 112 can be part of the input to the motor score prediction model 150.

The indicator metabolite trajectory 114 is a temporal trajectory of values of an indicator metabolite compound serving as a biomarker of disease activity of a disease that affects motor function. For example, the urinary biomarker hexose tetrasaccharide concentration (uHex4 (mmol/mol)) can serve as an indirect measure of the extent of skeletal muscle glycogen clearance in Pompe disease, which is correlated to a change in the motor function of the subject 110 having Pompe disease.

In some cases, the indicator metabolite trajectory 114 is obtained through laboratory measurement. For example, the uHex4 concentration values in urine samples of the subject can be measured at multiple time points for the subject 110. The uHex4 concentration values can be measured using any appropriate measurement techniques, such as tandem mass spectrometry (MS/MS) or enzymatic assays. The motor score prediction model 150 is configured to process the measured value of the indicator metabolite compound at each time point to predict the subject motor function score at that time point.

Clinical assessment of motor functions is time-consuming and requires specialized equipment and trained personnel. By contrast, the laboratory measurement of the indicator metabolite compound is generally much easier to access. Thus, using the measured indicator metabolite compound as a biomarker to infer the motor functions of the subject provides a more efficient and accessible means for monitoring the motor function changes in the subject 110. As described above, the motor score trajectory 140 predicted by the measured indicator metabolite trajectory 114 can be used to monitor the progression of the disease that affects motor function, determine the effectiveness of a treatment 170 administered to the subject 110, and optionally adjust the treatment strategy.

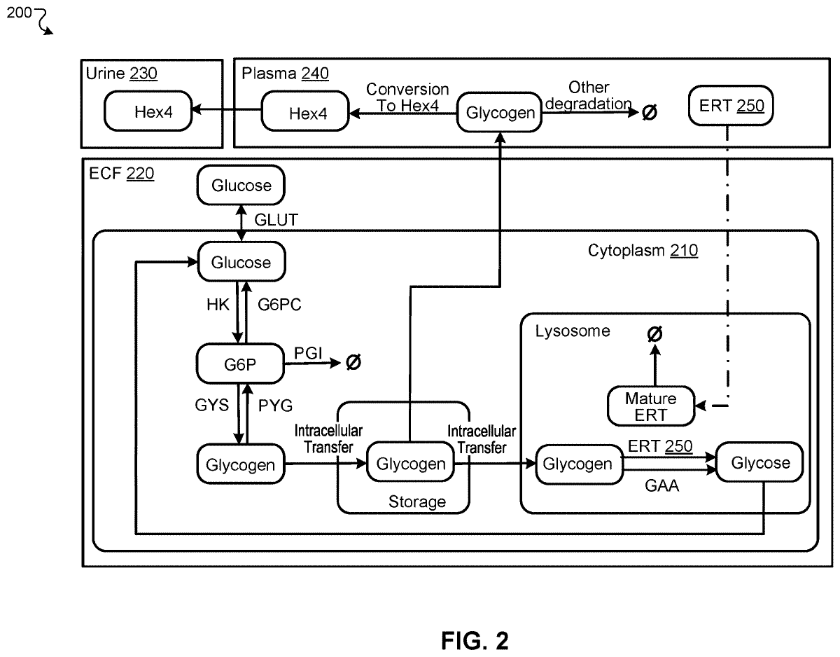

In some cases, instead of obtaining the indicator metabolite trajectory 114 through laboratory measurement, the system 100 or another system can predict the indicator metabolite trajectory 114 using a quantitative systems pharmacology (QSP) model 200 of the disease metabolism of a disease that affects the motor function. A particular example of the QSP model 200 for Pompe disease is described with reference to FIG. 2. In general, the QSP model for Pompe disease simulates the key processes underlying the disease. It can represent how the deficiency of the GAA enzyme leads to the buildup of glycogen within muscle cells, and predict changes in biomarkers like uHex4 for the particular subject 110. The QSP model 200 can also simulate enzyme replacement therapies. Thus, the system 100 or another system can use the QSP model to predict the uHex4 trajectory of a simulated subject that represents the real subject 110 having been diagnosed with a form of Pompe disease.

In particular, the QSP model 200 can be used to simulate an enzyme replacement therapy (which has not been administered to the real subject 110) for the simulated subject and predict the uHex4 trajectory of the subject under the enzyme replacement therapy. Thus, the QSP model 200 can be used to simulate different treatment scenarios (e.g., different treatment agents, doses, and/or administration timelines) and predict the uHex4 trajectories under the different treatment scenarios for the subject 110 without the subject 110 going through the treatments. The motor score trajectory 140 predicted from the simulated indicator metabolite trajectories (e.g., uHex4 trajectories) can then be used to assess how the real subject 110 would have responded to different therapies, and determine a selection of a particular treatment.

The motor score prediction model 150 is configured to, for each time point after a baseline time point (e.g., the first time point) of the indicator metabolite trajectory 114, process the indicator metabolite value at the time point to generate a respective predicted value for a change in the motor function score from a baseline value of the motor function score. The motor score prediction model 150 has a set of modal parameters 155.

In general, the motor score prediction model 150 includes a non-linear functional term of a change of the respective value for the indicator metabolite compound at the respective time point from a baseline value of the indicator metabolite compound. In the case of Pompe disease, the non-linear functional term correlates with the clinical observation that there is a non-linear increase of motor functions with uHex4 decrease. For example, after treatment onset, changes from baseline in motor functions have been observed to continue improving over time, with larger improvements seen in earlier treatment periods. The changes have been observed to reach a “plateau” after stabilization of the uHex4 concentration change. In some cases, the non-linear functional term is a decaying exponential functional term.

In some cases, the motor score prediction model 150 is a non-linear random coefficient model that includes one or more random parameters. For example, a rate of change parameter in the non-linear functional term can be a random parameter. In these cases, the random parameters are defined with respective distributions. To compute the predicted motor score trajectory 140 for a particular subject in a particular instance, the random parameters are sampled from the respective pre-defined distributions, and the predicted motor scores are computed using the sampled values of the random parameters.

In some cases, the non-linear functional term is a decaying exponential functional term. The rate of decay in the decaying exponential functional term can be a random parameter, and an asymptote parameter of the exponential functional term can be a fixed parameter.

The exact form of the motor score prediction model 150 can be adjusted to describe a particular clinical scenario. In some cases, the predicted motor function score is determined further based on (i) the baseline score for the subject and (ii) a median baseline score obtained for a population of subjects. In some cases, the predicted motor function score is determined further based on (i) an age of diagnosis of the subject and (ii) a median age of diagnosis obtained for a population of subjects. In some cases, the predicted motor function score is determined further based on a median baseline value for the indicator metabolite compound obtained for a population of subjects. For example, the subject 110 can be a subject that has been diagnosed with a particular disease that affects motor functions, and the population of subjects includes subjects having been diagnosed with the particular disease and being under a particular treatment.

In an illustrative example, for a population (I) of subjects that have been diagnosed with the IOPD form of the of Pompe disease, the motor score prediction model 150 for predicting a change in the GMFM score (e.g., GMFM-88) for subject i at time point j can take the form of:

$$Δ GMF \text{?} = \text{?} + \text{?} b . {GMFM}_{1} - median \text{?} ) + \text{?} ( b . uHex {4}_{i} - median \text{?} ) + \text{?} ( Age . {diag}_{i} - median \text{?} ) + \text{?} ( b . Mdu \text{?} - median \text{?} ) + \text{?} ( {gender}_{i} ) + ( \text{?} + \text{?} * ( b . Mdu \text{?} - mediane \text{?} ) ) \times ( 1 - \text{?} ) + {ε}_{ij} .$$

$$\text{?} \text{indicates text missing or illegible when filed}$$

The parameters C_{I,0}-C_{I,7} are fixed parameters obtained for the population I, b. GMFMᵢ is the baseline value for the GMFM score for subject i, median_{I,gmfm} is the median value for the GMFM score for the population I, b. uhex4ᵢ is the baseline value for the uHex4 for subject i, median_{I,uHex4} is the median value for the uHex4 marker for the population I, Age. diagᵢ is the age of diagnosis for the subject i, median_{I,age} is the median age of diagnosis for the population I, b. Mdurᵢ is the duration of previous Myozyme (alglucosidase alfa) use for subject i at baseline, median_{I,Mdur} is the median duration of Myozyme for the population I, rateᵢ is a random parameter sampled from a first normal distribution with rateᵢ>0, εᵢⱼ is another random parameter sampled from a second normal distribution, and ΔuHex4_{i,j} is the change from the baseline value for uHex4 for subject i at time point j. The parameters (mean and standard deviation values) defining the first and second normal distributions are determined based on the population I.

In another illustrative example, for a population (L) of subjects that have been diagnosed with the LOPD form of the of Pompe disease, the motor score prediction model 150 for predicting a change in the QMFT score for subject i at time point j can take the form of.

$$Δ QMF \text{?} = \text{?} + \text{?} ( b . {QMFT}_{i} - median \text{?} ) + \text{?} ( b . uHex {4}_{i} - median \text{?} ) + \text{?} ( Age . {diag}_{i} - median \text{?} ) + \text{?} ( age . {infusion}_{i} - median \text{?} ) + \text{?} ( b . {BMI}_{i} - median \text{?} ) + \text{?} ( b . {CK}_{i} - median \text{?} ) + \text{?} ( b . {CREAT}_{i} - median \text{?} ) + \text{?} \times ( 1 - \text{?} ) + {ε}_{ij} .$$

$$\text{?} \text{indicates text missing or illegible when filed}$$

The parameters C_{L,0}-C_{L,8} are fixed parameters obtained for the population L, b. QMFTᵢ is the base line value for the QMFT score for subject i, median_{I,QMFT} is the median value for the QMFT score for the population L, b. uhex4ᵢ is the baseline value for the uHex4 for subject i, median_{L,uHex4} is the median value for the uHex4 marker for the population L, age. infusionᵢ is the age of the start of infusion for the subject i, median_{L,ageinf} is the median age of the start of infusion for the population L, b. BMIᵢ is body mass index (BMI) of subject i at baseline, median_{L,bmi} is the median BMI for the population L, b. CKᵢ is the creatine kinase (CK) measurement for the subject i, median_{L,CK} is the median CK measurement for the population L, b. CREATᵢ is the creatine measurement for the subject i, median_{L,CREAT} is the median creatine measurement for the population L, rateᵢ is a random parameter sampled from a third normal distribution with rateᵢ>0, εᵢⱼ is another random parameter sampled from a fourth normal distribution, and ΔuHex4_{i,j} is the change from the baseline value for uHex4 for subject i at time point j. The parameters defining the third and fourth normal distributions are determined based on the population L.

The parameters 155 of the motor score prediction model 150 can be determined by a parameter estimation system 160 based on benchmarking data 160 for a particular population of subjects, such as a population of patients that have been diagnosed with a particular form of Pompe disease (e.g., IOPD or LOPD). The benchmarking data 160 includes, for each of a set of subjects, (i) respective input data for the model that specifies a temporal trajectory of a change in an indicator metabolite compound for the respective subject, and (ii) respective output data that specifies an output temporal trajectory of the change in the motor function score for the respective subject. The benchmarking data can further include subject-specific data for each subject such as demographic attributes, diagnosis history, treatment, and clinical data.

The parameter estimation system 160 can use any appropriate technique to determine the model parameters for the prediction model based on the benchmarking dataset. The parameter estimation system 160 can perform an optimization process to identify an optimal set of model parameters 155 that describe the relationship between the biomarker changes and motor function changes in the benchmarking dataset. In some cases, the motor score prediction model 150 is a non-linear random coefficient model, and the model parameters 155 include parameters describing fixed effects (applying to the whole population) and parameters describing the distribution of random effects (among individuals). In some cases, the optimization can be performed using maximum likelihood estimation (MLE) which identifies the model parameters that maximize the probability of observing the motor function changes observed in the benchmarking data. The MLE technique often involves iterative methods, starting with an initial guess and refining the parameters until a good fit is achieved. In some cases, the optimization can be performed using Bayesian methods, which start with a prior distribution of the model parameters, and then update the priors by incorporating the benchmarking data. Bayesian approaches can be computationally intensive but allow for greater flexibility and the ability to include prior knowledge. Example techniques of computation can include the gradient descent algorithm, Newton-Raphson algorithm, and the expectation-maximization (EM) algorithm. The choice of algorithm can depend on factors such as model complexity, size of the dataset, and desired computational speed. In some cases, specialized statistical software can be used to implement these techniques.

FIG. 2 shows an example QSP model 200 for describing cellular metabolism in Pompe disease. In particular, the QSP model 200 describes molecular-level reactions in the cytoplasm 210 a representative cell, linked with the indicator metabolite hexose tetrasaccharide (Hex4) in the plasma 240 and the urine 230 through the extracellular fluid 220.

The QSP 200 model simulates the deficiency in acid alpha-glucosidase (GAA) activity which results in glycogen accumulation in affected tissues and observed elevation of key urine and plasma biomarkers (such as Hex4). The QSP model 200 further simulates the effects of an enzyme replacement therapy (ERT) 250 (such as alglucosidase alfa and avalglucosidase alfa) which result in decrease tissue-specific glycogen burden and observed Hex4 in the urine and the plasma.

The parameters of the QSP model 200 can be determined for a population that has been diagnosed with a particular form of Pompe disease (IOPD or LOPD). Multiple data sources can be used to determine the QSP model parameters, including, for example, pharmacodynamic data, prior clinical studies of ERT for Pompe disease, and the Pompe Registry.

As described with reference to FIG. 1, the QSP model 200 can be used by a motor function prediction system to predict the uHex4 trajectory of a simulated subject that represents the real subject having been diagnosed with a form of Pompe disease. In particular, the QSP model 200 can be used to simulate a particular enzyme replacement therapy for the simulated subject and predict the uHex4 trajectory of the subject under the enzyme replacement therapy. Thus, the QSP model 200 can be used to simulate different treatment scenarios (e.g., different treatment agents, doses, and/or administration timing) and predict the uHex4 trajectories under the different treatment scenarios for a real subject without the subject going through the treatments. The motor function prediction system can process the simulated uHex4 trajectories to predict the motor function score trajectories under these different treatment scenarios, which indicate how the real subject would have responded to different therapies. This process can be performed for a population of subjects to assess the effects of the different therapies, taking into account the heterogeneity across patient cohorts.

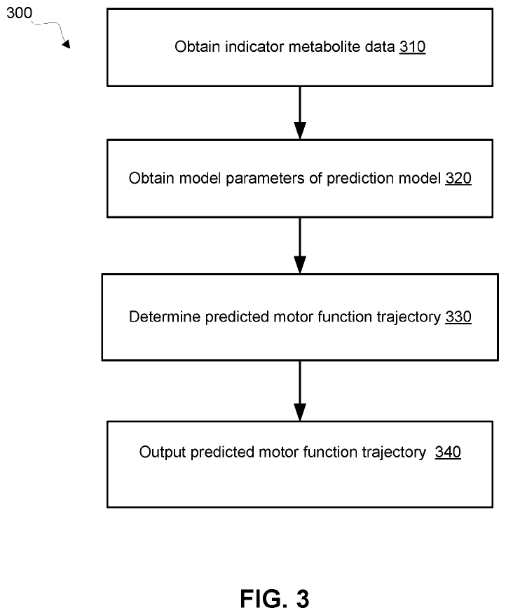

FIG. 3 is a flow diagram of an example process 300 for predicting a motor function of a subject. For convenience, the process 300 will be described as being performed by a system of one or more computers located in one or more locations. For example, a training system, e.g., the motor function prediction system 100 of FIG. 1, appropriately programmed in accordance with this specification, can perform the process 300.

At 310, the system obtains input data including a temporal trajectory of an indicator metabolite compound for the subject. The temporal trajectory of the indicator metabolite compound includes, for each of one or more time points, a respective value for the indicator metabolite compound for the subject at the respective time point. In some cases, the input data further includes (i) one or more demographic attributes of the subject, (ii) one or more clinical diagnosis for the subject, or (iii) a treatment history of the subject.

As described in further detail above with reference to FIG. 1, the subject can be a subject that has been diagnosed with a particular disease that affects motor functions, such as the Pompe disease (IOPD or LOPD). The subject can be under an enzyme replacement therapy for Pompe disease, such as an alpha-glucosidase replacement therapy or an avalglucosidase alfa replacement therapy.

In some cases, the temporal trajectory of the indicator metabolite compound can be obtained using laboratory tests. In some other cases, the temporal trajectory of the indicator metabolite compound can be generated using a simulation, such as using the QSP model. In particular, as described in further detail above with references to FIG. 1 and FIG. 2, in some cases, the subject is a simulated subject that represents real subject having been diagnosed with IOPD or LOPD, and a QSP model is used to predict the uHex4 trajectory. In some cases, the QSP model is used to simulate an enzyme replacement therapy (e.g., an alpha-glucosidase replacement therapy or an avalglucosidase alfa replacement therapy) for the simulated subject.

The QSP model parameters for a particular subject can be determined by adjusting one or more of a set of population-based model parameters based on one or more of: (i) one or more demographic attributes of the subject, (ii) one or more laboratory test results from the subject, (iii) one or more one clinical diagnosis for the subject, or (iv) a treatment history of the subject. As described in further detail above with reference to FIG. 2, the population-based model parameters can be obtained for a population that have been diagnosed with a particular form of Pompe disease (IOPD or LOPD). Multiple data sources can be used to determine the population-based QSP model parameters, including, for example, pharmacodynamic data, prior clinical studies of ERT for Pompe disease, and the Pompe Registry.

At 320, the system obtains a plurality of first model parameters of a motor function score prediction model. As described in further detail above with reference to FIG. 1, a parameter estimation system can obtain the first model parameters for the prediction model based on a benchmarking dataset that includes, for each of a plurality of subjects, (i) respective input data specifying a temporal trajectory of an indicator metabolite compound for the respective subject, and (ii) respective output data specifying a temporal trajectory of changes in the motor function score for the respective subject.

At 330, the system determines a predicted temporal trajectory of the motor function score. Examples of the motor function score include the GMFM score and the QMFT score. In particular, for each time point after a baseline time point in the temporal trajectory, the system determines a respective predicted value for a change in the motor function score from a baseline value at the respective time point using the prediction model based at least on (i) the input data and (ii) the first model parameters.

In general, the prediction model includes a non-linear functional term of a change of the respective value for the indicator metabolite compound at the respective time point from a baseline value. As described in further detail above with reference to FIG. 1, in some cases, the prediction model is a non-linear random coefficient model that includes one or more random parameters. For example, the prediction model can include a decaying exponential functional term, and the negative change rate parameter in the exponential functional term is a random parameter, while the asymptote parameter of the exponential functional term is a fixed parameter.

As described in further detail above with reference to FIG. 1, in addition to the indicator metabolite trajectory, the prediction model further takes into account additional attributes of the subject as well as the attributes for a population of subjects, such as: (i) the baseline score for the subject, (ii) a median baseline score obtained for the population of subjects, (iii) an age of diagnosis of the subject, (iv) a median age of diagnosis obtained for the population of subjects, (v) a median baseline value for the indicator metabolite compound obtained for the population of subjects, (vi) a treatment history of the subject, and (vii) additional laboratory test results for the subject. The population of subjects includes subjects having been diagnosed with the particular disease.

At 340, the system outputs data characterizing the predicted temporal trajectory of the motor function score. As described in further detail above with reference to FIG. 1, the predicted motor function score trajectory can be used in a variety of ways. For example, in some cases, the predicted motor function score trajectory can be used to determine the treatment efficacy of the enzyme replacement therapy for the subject. In some cases, the subject is a simulated subject that corresponds to a real subject, and the predicted motor function score trajectory for the simulated subject can be used to determine whether the real subject should receive the enzyme replacement therapy.

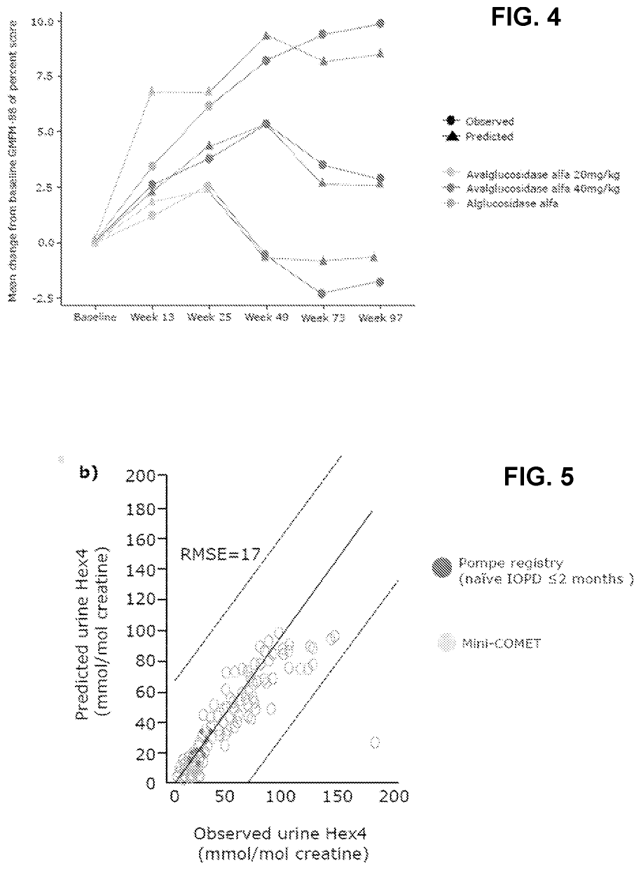

FIG. 4 illustrates the performance of the motor function score prediction model. The motor function score prediction model is used to predict the GMFM-88 trajectories of three cohorts of patients under different ERT therapies before week 25. All patients were switched to avalglucosidase alfa 40 mg/kg after week 25. The predicted GMFM-88 trajectories are compared with the observed GMFM-88 trajectories. In general, the predicted GMFM-88 trajectories are consistent with the trends of the observed trajectories, demonstrating the performance of the motor function score prediction model.

FIG. 5 illustrates the performance of QSP model. The uHex4 concentration predicted using the QSP model for a group of patients are compared with the observed uHex4 measurement. The comparison shows that the predicted uHex4 is in general consistent with the observed uHex4, demonstrating the performance of the QSP model.

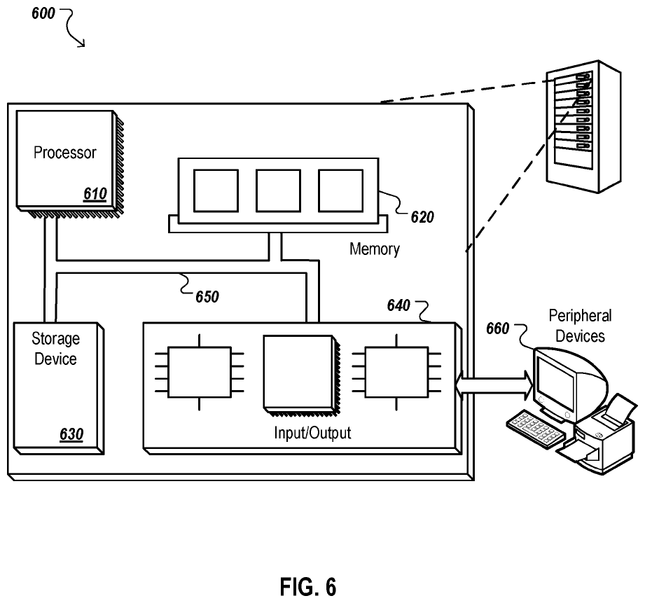

FIG. 6 is a block diagram of an example computer system 600 that can be used to perform operations described above. The system 600 includes a processor 610, a memory 620, a storage device 630, and an input/output device 640. Each of the components 610, 620, 630, and 640 can be interconnected, for example, using a system bus 650. The processor 610 is capable of processing instructions for execution within the system 600. In one implementation, the processor 610 is a single-threaded processor. In another implementation, the processor 610 is a multi-threaded processor. The processor 610 is capable of processing instructions stored in the memory 620 or on the storage device 630.

The memory 620 stores information within the system 600. In one implementation, the memory 620 is a computer-readable medium. In one implementation, the memory 620 is a volatile memory unit. In another implementation, the memory 620 is a non-volatile memory unit.

The storage device 630 is capable of providing mass storage for the system 600. In one implementation, the storage device 630 is a computer-readable medium. In various different implementations, the storage device 630 can include, for example, a hard disk device, an optical disk device, a storage device that is shared over a network by multiple computing devices (for example, a cloud storage device), or some other large capacity storage device.

The input/output device 640 provides input/output operations for the system 600. In one implementation, the input/output device 640 can include one or more network interface devices, for example, an Ethernet card, a serial communication device, for example, a RS-232 port, and/or a wireless interface device, for example, a 502.11 card. In another implementation, the input/output device can include driver devices configured to receive data and send output data to other input/output devices, for example, keyboard, printer and display devices 660. Other implementations, however, can also be used, such as mobile computing devices, mobile communication devices, set-top box television client devices, etc.

Although an example processing system has been described in FIG. 6, implementations of the subject matter and the functional operations described in this disclosure can be implemented in other types of digital electronic circuitry, or in computer software, firmware, or hardware, including the structures disclosed in this disclosure and their structural equivalents, or in combinations of one or more of them.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions.

Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, or a Jax framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment.

Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A computer-implemented method for predicting a motor function score measuring one or more motor functions of a subject, the method comprising:
obtaining input data comprising a temporal trajectory of an indicator metabolite compound for the subject, the temporal trajectory of the indicator metabolite compound comprising, for each of one or more time points, a respective value for the indicator metabolite compound for the subject at the respective time point;
obtaining a plurality of first model parameters of a prediction model;
determining a predicted temporal trajectory of the motor function score using the input data, the determining comprising: for each of the one or more time points after a baseline time point, determining a respective predicted value for a change in the motor function score from a baseline value at the respective time point using the prediction model based at least on (i) the input data and (ii) the first model parameters, wherein the prediction model includes a non-linear functional term of a change of the respective value for the indicator metabolite compound at the respective time point from a baseline value; and
outputting data characterizing the predicted temporal trajectory of the motor function score.

2. The method of claim 1, wherein the prediction model is a non-linear random coefficient model that comprises one or more random parameters.

3. The method of claim 2, wherein a rate of change parameter in the non-linear functional term is one of the random parameters.

4. The method of claim 2, wherein determining the respective predicted value comprises:
for each of the one or more random parameters, selecting a respective value for the respective random parameter from a respective pre-defined distribution;
wherein the predicted value is determined based on the respective values selected for the one or more random parameters.

5. The method of claim 1 wherein the non-linear functional term is a decaying exponential functional term.

6. The method of claim 5, wherein the prediction model is a non-linear random coefficient model, and a negative change rate parameter in the exponential functional term is a random parameter.

7. The method of claim 6, wherein an asymptote parameter of the exponential functional term is a fixed parameter.

8. The method of claim 1 wherein the motor function score is a Gross Motor Function Measure (GMFM) score or a Quality of Upper Extremity Skills Test (QMFT) score.

9. (canceled)

10. The method of claim 1, wherein the indicator metabolite compound is urinary hexose tetrasaccharide (uHex4), and the value for the indicator metabolite compound is a uHEX4 concentration value.

11. The method of claim 10, wherein one or more of the respective uHex4 concentration values are obtained through measurement of the uHex4 concentration at the respective time points for the subject.

12. The method of claim 1, wherein the input data further comprises (i) one or more demographic attributes of the subject, (ii) one or more clinical diagnosis for the subject, or (iii) a treatment history of the subject.

13. The method of claim 1, wherein the respective predicted value is determined further based on (i) the baseline score for the subject and (ii) a median baseline score obtained for a population of subjects.

14. The method of claim 1, wherein the predicted value is determined further based on (i) an age of diagnosis of the subject and (ii) a median age of diagnosis obtained for a population of subjects.

15. The method claim 1, wherein the respective predicted value is determined further based on a median baseline value for the indicator metabolite compound obtained for a population of subjects.

16. The method of claim 1, wherein the subject is a subject that has been diagnosed with a particular disease that affects motor functions.

17. The method of claim 16, wherein the particular disease is Infantile-onset Pompe disease (IOPD) or Late-onset Pompe disease (LOPD).

18. (canceled)

19. The method of claim 16, wherein the subject is under an enzyme replacement therapy for Pompe disease.

20. The method of claim 19, wherein the therapy is an alpha-glucosidase replacement therapy or an avalglucosidase alfa replacement therapy.

21. (canceled)

22. The method of claim 1, wherein obtaining the temporal trajectory of the indicator metabolite compound for the subject comprises:
obtaining a quantitative systems pharmacology (QSP) model of Pompe disease metabolism; and
predicting the temporal trajectory of the value of the indicator metabolite compound as a biomarker of disease activity using the QSP model at each of the one or more time points.

23.-48. (canceled)

49. A system comprising:
one or more computers; and
one or more storage devices storing instructions that when executed by the one or more computers, cause the one or more computers to perform operations for predicting a motor function score measuring one or more motor functions of a subject, the operations comprising:
obtaining input data comprising a temporal trajectory of an indicator metabolite compound for the subject, the temporal trajectory of the indicator metabolite compound comprising, for each of one or more time points, a respective value for the indicator metabolite compound for the subject at the respective time point:
obtaining a plurality of first model parameters of a prediction model:
determining a predicted temporal trajectory of the motor function score using the input data, the determining comprising: for each of the one or more time points after a baseline time point, determining a respective predicted value for a change in the motor function score from a baseline value at the respective time point using the prediction model based at least on (i) the input data and (ii) the first model parameters, wherein the prediction model includes a non-linear functional term of a change of the respective value for the indicator metabolite compound at the respective time point from a baseline value; and
outputting data characterizing the predicted temporal trajectory of the motor function score.

50. One or more computer-readable storage media storing instructions that, when executed by one or more computers, cause the one or more computers to perform operations for predicting a motor function score measuring one or more motor functions of a subject, the operations comprising:
obtaining input data comprising a temporal trajectory of an indicator metabolite compound for the subject, the temporal trajectory of the indicator metabolite compound comprising, for each of one or more time points, a respective value for the indicator metabolite compound for the subject at the respective time point;
obtaining a plurality of first model parameters of a prediction model;
determining a predicted temporal trajectory of the motor function score using the input data, the determining comprising: for each of the one or more time points after a baseline time point, determining a respective predicted value for a change in the motor function score from a baseline value at the respective time point using the prediction model based at least on (i) the input data and (ii) the first model parameters, wherein the prediction model includes a non-linear functional term of a change of the respective value for the indicator metabolite compound at the respective time point from a baseline value; and
outputting data characterizing the predicted temporal trajectory of the motor function score.
